(19) United States
(12) Patent Application Publication
(10) Pub. No.: US 2026/0007357 A1
(43) Pub. Date: Jan. 8, 2026

(54) DIAGNOSING NERUODEGENERATIVE DISEASES ASSOCIATED WITH DAMAGE TO THE ANTERIOR OLFACTORY NUCLEUS

(71) Applicants: Yeda Research and Development Co. Ltd., Rehovot (IL); Ichilov Tech Ltd., Tel-Aviv (IL)

(72) Inventors: Noam SOBEL, Jaffa (IL); Aharon WEISSBROD, Rehovot (IL); Danielle Ricca HONIGSTEIN, Rehovot (IL); Michal ANDELMAN GUR, Rehovot (IL); Tatyana GUREVICH, Tel-Aviv (IL); Adi EZRA, Tel-Aviv (IL); Neomi HEZI, Tel-Aviv (IL)

(73) Assignees: Yeda Research and Development Co. Ltd., Rehovot (IL); Ichilov Tech Ltd., Tel-Aviv (IL)

(21) Appl. No.: 19/259,027

(22) Filed: Jul. 3, 2025

Related U.S. Application Data
(60) Provisional application No. 63/667,180, filed on Jul. 3, 2024.

(51) Int. Cl. A61B 5/00; A61B 5/08; G16H 50/30

(52) U.S. Cl. CPC A61B 5/4082; A61B 5/0816; A61B 5/4011; A61B 5/7246; A61B 5/7264; G16H 50/30; A61B 2560/0242

(57) ABSTRACT

A method for generating an indication for Parkinson's Disease in a subject is disclosed. The method comprises measuring, whilst the subject is awake, air flow in the nose of the subject during a plurality of nasal respirations using a sensor; receiving, by a device, data associated with said air flow measurements received from said sensor; and evaluating at least one respiration parameter received from said data, generating an indication for Parkinson's Disease of the subject based on the at least one respiration parameter.

### RELATED APPLICATION(S)

This application claims the benefit of priority of U.S. Provisional Patent Application No. 63/667,180 filed on Jul. 3, 2024. The contents of the above applications are all incorporated by reference as if fully set forth herein in their entirety.

### FIELD AND BACKGROUND OF THE INVENTION

The present invention, in some embodiments thereof, relates to diagnosis and treatment monitoring for neurodegenerative diseases associated with damage to the anterior olfactory nucleus (AON) by measuring nasal respiration parameters.

Parkinson's Disease is an age-related disorder characterized by progressive loss of dopamine producing neurons in the substantia nigra of the midbrain, which in turn leads to progressive loss of motor functions manifested through symptoms such as tremor, rigidity and ataxia.

Current treatment strategies for PD focus on restoring the depletion of dopamine. generally through the administration of the dopamine precursor L-DOPA (L-3-4-dihydroxyphenylalanine). L-DOPA, (the blood-brain barrier (BBB) penetrating precursor of dopamine), successfully increases the synthesis and release of dopamine. However, as the disease progresses, less dopaminergic neurons are available to synthesize dopamine from the precursor and the effectiveness of the treatment decreases whilst L-DOPA-induced dyskinesia appears. Other treatments with dopamine agonists, monoamine oxidants inhibitor or COMT inhibitors also demonstrate partial improvement but they cannot prevent progression of the disease.

The peripheral pulmonary findings in PD are assorted, and include altered function in respiratory muscles and the expected ensuing reduced lung volumes, reduced maximal inspiratory and expiratory flows, and diaphragmatic dyskinesias. Indeed, the ultimate cause of death in PD is often aspiration pneumonia. In contrast to this extensive investigation on the peripheral mechanisms of breathing in PD, there has been limited investigation on the central mechanisms of breathing in PD.
Additional background art includes: Bhatia-Dey, N.; Heinbockel, T. Int. J. Environ. Res. Public Health 2021, 18, 6976. https://doi(dot)org/10.3390/ijerph18136976; Attems et al., Acta Neuropathol (2014) 127:459-475;
Yang, Y., Yuan, Y., Zhang, G. et al. Nat Med 28, 2207-2215 (2022). https://doi(dot)org/10.1038/s41591-022-01932.
US patent publication 2023/0028914A1;
PCT Application No. WO2021/209999; and
PCT Application No. WO2023/209712.

### SUMMARY OF THE INVENTION

Following is a non-exclusive list including some examples of embodiments of the invention. The invention also includes embodiments which include fewer than all the features in an example and embodiments using features from multiple examples, also if not expressly listed.

According to an aspect of the invention, there is provided a method for generating an indication for Parkinson's Disease in a subject comprising:
measuring, whilst the subject is awake, air flow in the nose of the subject during a plurality of nasal respirations using a sensor;
receiving, by a device, data associated with the air flow measurements received from the sensor;
evaluating at least one respiration parameter received from the data, wherein the respiration parameter comprises at least one of a duration of a breath, a variability of respiration rate and a pausing characteristic of the breath; and
generating an indication for Parkinson's Disease of the subject based on the at least one respiration parameter.

According to embodiments of the invention, the measuring comprises measuring air flow in the at least one nostril during at least two consecutive nasal respirations of the subject.

According to embodiments of the invention, the measuring comprises measuring airflow in the at least one nostril during at least 10% of the breaths of the subject over a period of at least ten minutes.

According to embodiments of the invention, the evaluating is affected using data which is retrieved no later than the first hour of measuring.

According to embodiments of the invention, the evaluating is effected using data which is retrieved whilst the subject is carrying out a predefined activity.

According to embodiments of the invention, the method further comprises measuring, a time between two nasal inhalations, an inhalation velocity, an exhalation velocity and a time between two nasal exhalations.

According to embodiments of the invention, the evaluating comprises evaluating one or both of a coefficient of variation of the parameter over a predetermined time period and a peak value of the parameter over a predetermined time period.

According to embodiments of the invention, the measuring comprises measuring airflow in two nostrils and the at least one respiration parameter is evaluated from measurements from both the two nostrils.

According to embodiments of the invention, the generating the indication comprises applying a pre-trained classifier on the at least one respiration parameter.

According to embodiments of the invention, the generating an indication comprises determining whether the subject has Parkinson's Disease or is at risk of having Parkinson's Disease.

According to embodiments of the invention, the duration of a nasal breath is a duration of a nasal inspiration.

According to embodiments of the invention, the duration of a nasal breath is a duration of a nasal expiration.

According to embodiments of the invention, the generating an indication comprises determining a severity of the Parkinson's disease.

According to embodiments of the invention, the characteristic is a proportion of breaths with inhale pause and/or a proportion of breaths with exhale pause.

According to another aspect of the invention there is provided a method for generating an indication for Alzheimer's disease (AD) in a subject comprising:
measuring air flow in the nose of the subject during a plurality of nasal respirations using a sensor;
receiving, by a device, data associated with said air flow measurements received from said sensor;
extracting at least one respiration parameter from the data, wherein said respiration parameter is affected by exhalation;
evaluating at least one said respiration parameter extracting from said data; and
generating an indication for AD of the subject based on said at least one respiration parameter.

According to embodiments of the invention, the generating an indication comprises determining whether the subject has AD or is at risk of having AD.

According to embodiments of the invention, the generating an indication comprises determining a severity of said AD.

According to embodiments of the invention, the respiration parameter comprises a duration of a nasal exhalation, an exhalation velocity and a time between two nasal exhalations.

According to an aspect of the invention, there is provided a method for generating an indication for a severity of a neurodegenerative disease associated with damage to the anterior olfactory nucleus (AON) in a subject comprising:
measuring, whilst the subject is awake, air flow in the nose of the subject during a plurality of nasal respirations using a sensor;
receiving, by a device, data associated with the air flow measurements received from the sensor;
evaluating at least one respiration parameter received from the data; and
generating an indication for the severity of the neurodegenerative disease associated with damage to the AON of the subject based on the at least one respiration parameter.

According to embodiments of the invention, the at least one respiration parameter comprises a temporal respiration parameter.

According to embodiments of the invention, the temporal respiration parameter comprises at least one of a duration of a nasal inhalation, a duration of a nasal exhalation, a time between two nasal inhalations, an inhalation velocity, an exhalation velocity and a time between two nasal exhalations.

According to embodiments of the invention, the evaluating comprises evaluating one or both of a coefficient of variation of the parameter over a predetermined time period and a peak value of the parameter over a predetermined time period.

According to embodiments of the invention, the neurodegenerative disease associated with damage to the AON is selected from the group consisting of Parkinson's Disease (PD) and Alzheimer's disease (AD).

According to embodiments of the invention, the neurodegenerative disease associated with damage to the AON is PD.

According to embodiments of the invention, the temporal respiration parameter comprising at least one of a breathing rate variation coefficient, a duty cycle inhale and a duty cycle of inhale pause duration.

According to embodiments of the invention, the neurodegenerative disease associated with damage to the AON is selected from the group consisting of Parkinson's Disease (PD), Alzheimer's disease (AD).

According to embodiments of the invention, the neurodegenerative disease associated with damage to the AON is AD.

According to embodiments of the invention, the temporal respiration parameter comprises at least one of a duration of a nasal exhalation, an exhalation velocity and a time between two nasal exhalations.

According to embodiments of the invention, the neurodegenerative disease associated with damage to the AON is PD.

According to another aspect of the invention, there is provided a system for generating an indication for Parkinson's Disease in a subject comprising:
receiving circuitry configured to receive a measurement signal including measurements of a plurality of respirations from a sensor configured to sense a respiration of the subject whilst awake;
determining circuitry configured to determine values of one or more respiration parameter from the measurement signal, the respiration parameter comprises at least one of a duration of a breath, a variability of respiration rate and a pausing characteristic of the breath;
evaluating circuitry configured to diagnose the subject, based on the values of one or more respiration parameter to provide an indication of the Parkinson's Disease.

According to embodiments of the invention, the sensor comprises a nasal flow sensor.

According to embodiments of the invention, the one or more respiration parameter comprises a temporal nasal respiration parameter.

According to embodiments of the invention, the sensor comprises a pressure sensor.

According to embodiments of the invention, the evaluation circuitry includes a classifier trained to translate breathing data into a Parkinson's Disease diagnosis.

According to embodiments of the invention, the Parkinson's Disease diagnosis comprises a disease sub-classification or a disease severity score.

According to embodiments of the invention, the evaluation circuitry is configured to compare the value of the at least one respiration parameter with a reference value, wherein a difference between the value of at least one respiration parameter of the subject and the reference value is indicative of the disease diagnosis.

According to embodiments of the invention, the system comprises memory storing therein at least one reference value personalized for the subject.

According to embodiments of the invention, the determining circuitry or the evaluating circuitry is remote from the receiving circuitry.

According to embodiments of the invention, the system comprises at least one additional sensor configured to sense environmental data and/or physiological data of the subject and wherein the evaluation circuitry is configured to take such data into account in the evaluating.

According to another aspect, there is provided a system for generating an indication of severity of a neurodegenerative disease associated with damage to the anterior olfactory nucleus (AON) in a subject comprising:
receiving circuitry configured to receive a measurement signal including measurements of a plurality of respirations from a sensor configured to sense a respiration of the subject whilst awake;
determining circuitry configured to determine values of one or more respiration parameter from the measurement signal;
evaluating circuitry configured to diagnose the subject, based on the values of one or more respiration parameter to provide an indication of severity of the neurodegenerative disease associated with damage to the AON.

According to embodiments of the invention, the sensor comprises a nasal flow sensor.

According to embodiments of the invention, the one or more respiration parameter comprises a temporal nasal respiration parameter.

According to embodiments of the invention, the temporal respiration parameter comprises at least one of a duration of a nasal inhalation, a duration of a nasal exhalation, a time between two nasal inhalations, an inhalation velocity, an exhalation velocity and a time between two nasal exhalations.

According to embodiments of the invention, the temporal respiration parameter comprising at least one of a breathing rate variation coefficient, a duty cycle inhale and a duty cycle of inhale pause duration

According to embodiments of the invention, the sensor comprises a pressure sensor.

According to embodiments of the invention, the evaluation circuitry includes a classifier trained to translate breathing data into a neurodegenerative disease subclassification or a disease severity score.

According to embodiments of the invention, the evaluation circuitry is configured to compare the value of the at least one respiration parameter with a reference value, wherein a difference between the value of at least one respiration parameter of the subject and the reference value is indicative of the disease diagnosis.

According to embodiments of the invention, the system comprises memory storing therein at least one reference value personalized for the subject.

According to embodiments of the invention, the determining circuitry or the evaluating circuitry is remote from the receiving circuitry.

According to embodiments of the invention, the system comprises at least one additional sensor configured to sense environmental data and/or physiological data of the subject and wherein the evaluation circuitry is configured to take such data into account in the evaluating.

According to embodiments of the invention, the neurodegenerative disease is selected from the group consisting of Parkinson's Disease (PD), Alzheimer's disease (AD)

According to still another aspect there is provided a method of determining whether a therapy or therapeutic agent is effective in treating Parkinson's Disease in a subject comprising:
applying the therapy to the subject or administering the therapeutic agent to the subject; and
determining a status of the subject's Parkinson's Disease based on a plurality of nasal respirations of the subject whilst awake, wherein a similarity of one or more nasal respiration parameter ascertained from the nasal respirations to a nasal respiration parameter ascertained from nasal respirations of a healthy subject whilst awake is indicative of an efficacious therapy or therapeutic agent

According to embodiments of the invention, the respiration parameter comprises at least one of a duration of a breath, a variability of respiration rate and a pausing characteristic of the breath.

According to embodiments of the invention, the subject has received dopamine or a dopamine analogue on the day of the determining.

According to embodiments of the invention, the applying comprises indicating such applying or administering to a system used to measure the disease status.

According to still another aspect, there is provided a computer readable media having thereon a classifier for Parkinson's Disease based on nasal breathing parameters whilst the subject is awake.

According to still another aspect, there is provided a method of creating a classifier for Parkinson's Disease based on nasal breathing respiration parameters of the subject, comprising:
measuring respirations of a subject whilst awake; and
collecting data regarding nasal respiration parameters from the recording; and
training a machine learning system with the data using an indication of a known Parkinson's Disease diagnosis of the subject.

According to embodiments of the invention, the nasal respiration parameters comprise at least one of a duration of a breath, a variability of respiration rate and a pausing characteristic of the breath.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as assessing a subject, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIGS. 1A-D: A wearable logger of nasal airflow. A. The Nasal Holter device pasted onto the nape of a participant's neck. B. The stereo nasal cannula communicating a pressure wave from each nostril separately. C-D. Examples of the nasal respiratory trace obtained from (C) a healthy control and (D) a PD patient.

FIGS. 2A-C. Significantly altered nasal airflow in PD across time during wake. Line plots of nasal airflow parameters during wake from starting point of the recording, for PD patients and healthy controls. A. Duty Cycle inhale. B. Breathing rate variation coefficient. C. Duty cycle inhale pause. Solid line is the mean, and shade represents SEM. n(control)=33, n(PD)=28. The plot is restricted to the duration of the one patient that provided least wake data (390 minutes). Overall wake recordings ranged from 390 to 1135 minutes (mean=868.5±147.7).

FIGS. 3A-D. Significantly altered nasal airflow in PD along hour of day during wake. Line plots of nasal airflow parameters during wake along hour of day, for PD patients and healthy controls. A. Duty Cycle inhale. B. Breathing rate variation coefficient. C. Duty cycle inhale pause. Solid line is the mean, and shade represents SEM. D. Histogram showing the number of participants from each group for each hour along the day.

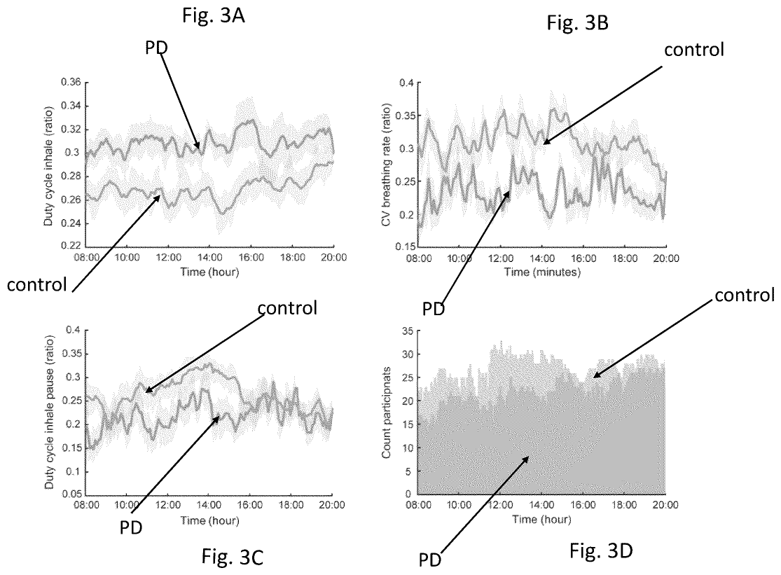

FIGS. 4A-F: Classifying PD from nasal airflow during wake. A-C. Box plots for respiration parameters during the first 30-min of recording during wake, n(control)=33, n(PD)=28. A. Duty Cycle inhale. B. Breathing rate variation coefficient. C. Duty cycle inhale pause. Central lines indicate median, top and bottom edges of the box indicate 75th and 25th percentiles, respectively. Whiskers reflect 1.5 times the interquartile range. * P≤0.05, ** P≤0.01, *** P≤0.001. D. ROC curve for detecting PD from 5 minutes (0.83) 30 minutes (0.85), and 6.5 hours (0.76) using a subspace discriminant classifier (n(control)=33, n(PD)=28). E. Histogram of classification accuracy for shuffled data with actual classification accuracy marked by dashed line. F. Model accuracy over time.

FIGS. 5A-D. Nasal airflow parameters during sleep. A-C. Box plots of nasal airflow parameters during the first 30-min of recording during sleep, n(control)=33, n(PD)=28. A. Duty Cycle inhale (mean PD=0.34±0.11, mean Control=0.34±0.06, Wilcoxon rank-sum test, z=−0.56, effect size Cliff's d=−0.08, P=NS). B. Duty cycle inhale pause (mean PD=0.1±0.09, mean Control=0.1±0.1, Wilcoxon rank-sum test, z=0.22, effect size Cliff's d=0.03, P=NS). C. Breathing rate coefficient of variance (mean PD=0.25±0.18, mean Control=0.19±0.13, Wilcoxon rank-sum test, z=1.32, effect size Cliff's d=0.2, P=NS). All presented P values were corrected for multiple comparisons using Bonferroni correction. The central line indicates median, and the top and bottom edges of the box indicate 75th and 25th percentiles, respectively. Whiskers reflect 1.5 times the interquartile range. D. Model AUC across sampling time during sleep, showing non-significant results with AUC around 0.49. The model was implemented on the first hour of nocturnal nasal respiration, which was equivalent to the shortest recording interval during sleep.

FIGS. 6A-I. Nasal airflow parameters during wake in various disease duration sub-cohorts. Box plots of nasal airflow parameters during the first 30-min of recording during wake in various disease duration sub-cohorts. A-C. Disease duration ≤20 years, n(control)=33, n(PD)=27, D-F. Disease duration £10 years, n(control)=33, n(PD)=23, G-I. Disease duration £5 years, n(control)=33, n(PD)=12. * P≤0.05, ** ≤£ 0.01, *** P≤0.001. Statistics were calculated using Wilcoxon Rank sum test. All presented P values were corrected for multiple comparisons using Bonferroni correction. The central line indicates median, and the top and bottom edges of the box indicate 75th and 25th percentiles, respectively. Whiskers reflect 1.5 times the interquartile range. A. Duty cycle inhale: mean PD=0.3±0.07, mean control=0.23±0.06, z=4.2, P=7.3×10−5. B. Breathing rate variation coefficient: mean PD=0.26±0.1, mean control=0.38±0.1, z=−4.26, P=6.0×10−5. C. Duty Cycle inhale pause: mean PD=0.29±0.1, mean control=0.21±0.3, z=−2.9, P=0.01. D. Duty cycle inhale: mean PD=0.3±0.07, mean control=0.23±0.06, z=4.2, P=8.7×10−5. E. Breathing rate variation coefficient: mean PD=0.26±0.1, mean control=0.38±0.1, z=−3.9, P=0.0002. F. Duty Cycle inhale pause: mean PD=0.2±0.1, mean control =0.29±0.1, z=−3.01, P=0.007. G. Duty cycle inhale: mean PD=0.29±0.08, mean control=0.23±0.06, z=2.8, P=0.02. H. Breathing rate variation coefficient: mean PD=0.26±0.1, mean control=0.38±0.1, z=−3.5, P=0.001. I. Duty cycle inhale pause: mean PD=0.2±0.1, mean control=0.29±0.1, z=−2.6, P=0.03.

FIGS. 7A-E. Classifier results in various disease duration sub-cohorts. ROC curve plots for detecting PD from 30 minutes of wake using a subspace discriminant classifier, with data from patients with various disease durations. To create even-numbered groups of PD and control for each disease duration category, control group was matched based on age and gender criteria. A. Disease duration ≤20 years, n(control)=27, n(PD)=27, sensitivity: 22 of 27 (81.5%), specificity: 22 of 27 (81.5%), accuracy 44 of 54 (81.5%), AUC 0.87. B. Disease duration ≤10 years, n(control)=23, n(PD)=23, sensitivity: 19 of 23 (82%), specificity: 19 of 23 (82%), accuracy 38 of 46 (82%), AUC 0.87. C. Disease duration≤5 years, n(control)=12, n(PD)=12, sensitivity: 10 of 12 (83%), specificity: 8 of 12 (66%), accuracy 18 of 24 (75%), AUC 0.81. D. Disease duration <10 years, n(control)=5, n(PD)=5, sensitivity: 3 of 5 (60%), specificity: 4 of 5 (80%), accuracy: 7 of 10 (70%), AUC 0.48. E. Disease duration 5-10 years, n(control)=11, n(PD)=11, sensitivity: 9 of 11 (82%), specificity: 10 of 11 (91%), accuracy: 19 of 22 (86%), AUC 0.9;

FIGS. 8A-E. Predicting PD severity from nasal airflow. Correlation between Severity prediction and actual Movement Disorder Society-Unified Parkinson's Disease Rating Scale (MDS-UPDRS) A. Total score. B. Part I. C. Part II. D. Part III. E. Part IV. Each dot represents a participant (n=28, jittered to prevent overlay). The light patch represents 95% confidence interval;

FIG. 9 is a flow chart of a process for determining status of neurodegenerative disease;

FIG. 10 is a flow chart of a process for determining responsiveness of a subject to a treatment for Parkinson's Disease, according to some exemplary embodiments of the invention;

FIG. 11 is a block diagram of a system for determining status of Parkinson's Disease, according to some exemplary embodiments of the invention;

FIGS. 12A-C are box plots illustrating that inhales are longer and less variant in PD compared to healthy subjects. Box plots are illustrative for respiration parameters during the first 25-min of recording during wake, n(control)=33, n(PD)=28, n (AD)=17. A. Breathing rate variation coefficient. B. Duty Cycle inhale. C. Duty cycle inhale pause. Central lines indicate median, top and bottom edges of the box indicate 75th and 25th percentiles, respectively. Whiskers reflect 1.5 times the interquartile range. * P<0.05, ** P<0.01, *** P<0.001.

FIGS. 13A-B are box plots illustrating that exhales are longer in AD. Box plots are illustrative for respiration parameters during the first 25-min of recording during wake, n(control)=33, n(PD)=28, n (AD)=17. A. Duty Cycle exhale. B. Exhale Duration. Central lines indicate median, top and bottom edges of the box indicate 75th and 25th percentiles, respectively. Whiskers reflect 1.5 times the interquartile range. * P<0.05, ** P<0.01, *** P<0.001.

FIGS. 14A-B are graphs illustrating that exhale duration is correlated with MoCA Score. Correlation between MoCA Score and A. Duty Cycle Exhale and B. Exhale Duration. Spearman's r and P values are indicated.

### DESCRIPTION OF SPECIFIC EMBODIMENTS OF THE INVENTION

The present invention, in some embodiments thereof, relates to diagnosis and treatment monitoring for neurodegenerative diseases associated with damage to the anterior olfactory nucleus (AON) by measuring nasal respiration parameters. Such methods may be used for, for example, providing objective markers of the neurodegenerative diseases that might be used for diagnosis, staging, prognosis and/or assessment of response to pharmacological interventions.

A broad aspect of some embodiments relates to determining the status (i.e., determining an indication) of a neurodegenerative disease associated with a damage to anterior olfactory nucleus (AON) of a subject based on nasal respiration measurements.

The present inventors have shown that values and/or changes in values of respiration parameters (e.g., nasal respiration parameters) are indicative of neurodegenerative disease status (e.g., severity). Analysis of these parameters allow the user to obtain feedback (e.g., substantially instantaneous feedback) regarding disease status.

Neurodegenerative diseases which are associated with damage to anterior olfactory nucleus (AON) include but are not limited to Parkinson's disease, Alzheimer's Disease.

In some embodiments, the measuring of nasal respiration parameters is carried out using a portable/mobile device that is inserted inside or close to at least one nostril of the subject. The device may be retained at the site of measurement and/or on the subject's body without additional equipment or personnel, e.g., using a strap, a halter etc. In some embodiments of the invention, the device and/or other system components carried by the subject are mobile, for example, weighing less than 1 kg, 500 gr and smaller or intermediate weights.

In some embodiments, measuring of oral respiration parameters is also considered.

Devices for measuring oral airflow are known in the art and include for example, using a pneumotachometer and a mouthpiece.

Alternatively or additionally, in some embodiments, airflow is measured by measuring movement of the abdomen during respiration, exemplary sensors being piezoelectric elements responding to changes in length associated with respiration of the subject.

Alternatively or additionally, in some embodiments, temperature of nasal airflow is measured, for example by thermistor/s placed within the nasal air flow (and e.g., not in contact with the skin). Alternatively or additionally, in some embodiments, a pneumotachometer is used to measure differential pressure of the nasal air flow. Where, in some embodiments, the differential pressure is converted into a voltage signal using a spirometer.

Optionally or additionally, some of the components are dual use, for example, a user-interface (UI) and/or some processing and/or communication functionality are provided by a mobile telephone or other mobile device used by the subject for other activities. The device itself may comprise circuitry which analyzes parameters of the respiration (e.g., nasal) signals and optionally further comprises circuitry that communicates the status of the neurodegenerative disease based on values of these parameters. Alternatively, the device may be dedicated to measuring nasal respiration signals (e.g., nasal airflow) and may comprise circuitry such that it is connectable to an external processor/processors (e.g., on a mobile device such as a mobile telephone) which carry out the function of analysis and/or status communication. A potential benefit of using such a device (and optional mobile processor) to assess neurodegenerative status of a subject is the relative simplicity of equipment and/or testing technique/s required e.g., as opposed to imaging methods e.g., structural and/or functional brain imaging e.g., neuroimaging and electrophysiology. Since measurement of respiratory parameters whilst the subject is awake was shown to be sufficient for providing an indication of neurodegenerative disease status for a relatively short amount of time (e.g., 30 minutes), the subject is able to monitor disease status on a frequent basis. In one embodiment, the subject is stationary during the measurement. In another embodiment, the subject performs a predetermined task (i.e., activity) during the measurement.

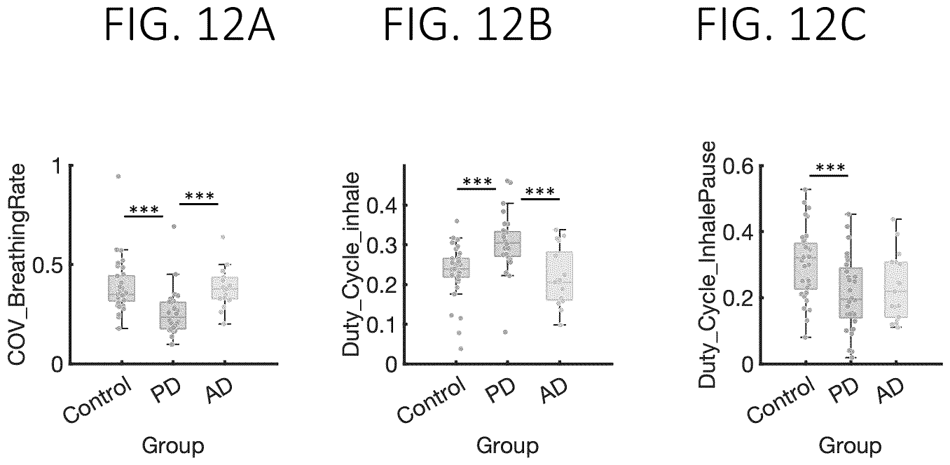

The present inventors showed that they were able to diagnose Parkinson's disease even when the patient was taking medication to dampen the clinical manifestations of the disease (e.g., on dopamine treatment). The ability to determine disease status under these conditions, allows for testing of additional drugs which may affect disease etiology e.g., in a personalized fashion. The present inventors further showed that both nasal inhalation and nasal exhalation data could be used to diagnose Parkinson's. For Alzheimer's Disease, nasal exhalation data was more indicative (see FIGS. 12A-C and 13A-B).

In some embodiments of the invention, the device includes a user-interface allowing a subject to enter information (such as taking of drugs) and/or allowing the system to provided neurodegenerative disease status and/or other guidance. Optionally, the UI is used to carry out various interactive methods as described herein, where the subject performs some activities (e.g., data entry) and the processor others (e.g., determining neurodegenerative disease state). The device may store personalized attributes of the subject and/or include sensors to collect environmental and other data. As noted, some functions of the device may be carried out by associated devices of a system for neurodegenerative disease subjects, for example, processing, UI and/or sensing may be provided by a cellular telephone. Optionally or additionally, processing may be provided by a remote server (e.g., a cloud server).

In some embodiments, assessment of neurodegenerative disease status of a subject using nasal respiration parameter/s is performed repetitively over time, for example, to assess subject progression over time. For example, to assess effectiveness of treatment e.g., over time, for example, as further described below. In this context, over time includes over a treatment session, for example detecting that efficacy of a drug is wearing off and/or over one or more days, weeks or months, to track progress of a subject.

In some embodiments, nasal respiration parameter monitoring are performed repetitively, e.g., to provide a log of patient progress, optionally along with treatment data.

In accordance with some embodiments of the invention, there is provided a method for processing physiological data, comprising: (a) collecting nasal respiration data from a subject; (b) using a pre-trained machine learning model to process the nasal respiration data to obtain a processed signal; and (c) using the processed signal to identify a neurodegenerative disease status of the subject.

In some embodiments of the invention, the methods described herein for tracking neurodegenerative disease status are used over a period of 1-5 months or years, or longer. The improvement or worsening of disease score and/or response to medication may be used for prognosis, for example, based on prognosis of patients with similar etiologies and/or based on a monotonic (e.g., things will continue improving or worsening) or an asymptotic (e.g., if change is getting smaller, change will plateau and an asymptote will be reached) assumption.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Exemplary General Process for Determining Status of Neurodegenerative Disease

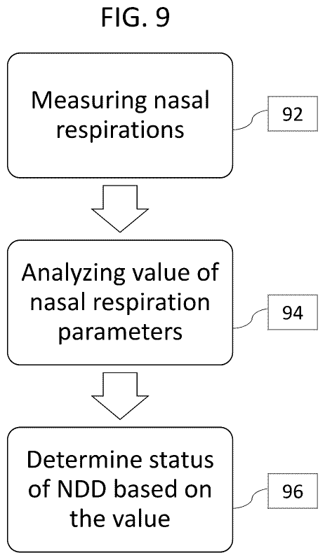

Reference is now made to FIG. 9, depicting a process for determining a neurodegenerative status of a subject, (and optionally predicting a future disease status), according to some exemplary embodiments of the invention.

According to some exemplary embodiments, nasal respirations (e.g., nasal airflow) are recorded at block 92. In some embodiments, additional respiratory signals are recorded from the mouth.

In some embodiments, respiration is measured using one or more movement sensor and/or pressure sensor and/or one or more optical sensor.

For example, in some embodiments, a pressure sensor senses changes in pressure on the sensor associated with respiration. In some embodiments, pressure sensor is held in contact with and/or in position close to at least one nostril of the subject e.g., by a strap and/or adhesive.

Exemplary devices for measuring nasal respiration are known in the art and some are described in more detail herein below. However, other devices for measuring nasal respirations may be used as well.

In some embodiments of the invention, the measuring is carried out whilst the subject is awake.

In some embodiments, the measuring is carried out whilst the subject is stationary.

In some embodiments, the measuring is carried out whilst the subject is performing a predefined activity—e.g., reading, speaking, movement etc.

The subject may be of any age, including children (e.g., under the age of 18). Typically, the subject is between 40 and 80 years old.

In one embodiment, a plurality of nasal respirations (e.g., of a same nostril) are measured. The nasal respiration may be measured at a single nostril or both nostrils. In one embodiment, the airflow in one nostril is measured independently of the airflow in the second nostril. In one embodiment at least two consecutive nasal respirations are recorded. In another embodiment at least 3, 4, 5, 6, 7, 8, 9, or 10 nasal respirations (optionally consecutive) are recorded. For example, measurement of 1-100 respirations, or 10-100 respirations, or 50-100 respirations, 50-500 respirations, or 1-30 minutes, or 1-20 minutes, or 5-30 minutes. In one embodiment, nasal respiration measurements are taken for no longer than one hour. It is noted that some nasal measurements may be skipped or not collected. For example, measurement may be on a sampling basis. Optionally or additionally, an accelerometer or other sensor (e.g., as will be described below) may be used to detect movement of the subject and such measurements ignored. Similarly, heart rate may be used as an indication to ignore some measurements or otherwise process them differently, for example, heart rate changes indicating an increase in activity.

It is a particular feature of some embodiments of the invention that as evaluation of neurodegenerative disease status is rapid, data can be skipped in a more cavalier fashion and still result in rapid and/or quality evaluation. For example, fewer than 80%, 60%, 50%, 30%, 15% or 10% of breathing cycles may be measured and/or utilized in a time period (e.g., with at least 5, etc. respiration used).

In some embodiments, one or more nasal respiration parameter is determined continuously e.g., using continuous respiration measurements. In some embodiments, the subject is assessed using these respiration parameter/s continuously. Alternatively or additionally, in some embodiments, continuous respiration measurements are used to assess the subject periodically.

It is a particular feature of some embodiments of the invention, the neurodegenerative disease status (and/or a change therein) can be determined within a time window of, for example, less than 30 minutes, less than 20 minutes, less than 15 minutes, less than 10 minutes, about or less than 5 minutes or shorter or intermediate times. This potentially allows rapid feedback to therapeutic processes and/or to obtain a finer grained understanding of a subject's disease journey and/or potentially predictive methods.

In some embodiments, a subject is assessed using measured nasal respiration parameters periodically. In some embodiments, respiration is measured (or continuous measurements are sampled) for short periods of time e.g., on a regular basis, for example, for 5-30 mins.

It will be appreciated that the number of measurements and/or length of time during which the nasal respirations are recorded may be adapted according to the disease status (and particular disease) that is being measured.

In one embodiment, the measuring is affected in at least 10%, 20%, 30%, 40%, 50%, 60%, 70%, 80%, 90% or even 100% of all breaths over a time period of at least five minutes.

In one embodiment, the measuring is affected in at least 10%, 20%, 30%, 40%, 50%, 60%, 70%, 80%, 90% or even 100% of all breaths over a time period of at least ten minutes.

In one embodiment, the measuring is affected in at least 10%, 20%, 30%, 40%, 50%, 60%, 70%, 80%, 90% or even 100% of all breaths over a time period of at least twenty minutes.

In one embodiment, the measuring is affected in at least 10%, 20%, 30%, 40%, 50%, 60%, 70%, 80%, 90% or even 100% of all breaths over a time period of at least thirty minutes.

In one embodiment, the measuring is affected in at least 10%, 20%, 30%, 40%, 50%, 60%, 70%, 80%, 90% or even 100% of all breaths over a time period of at least one hour.

In one embodiment, the measuring is affected in at least 10%, 20%, 30%, 40%, 50%, 60%, 70%, 80%, 90% or even 100% of all breaths over a time period of at least 2-10 hours.

In still further embodiments, the measuring is affected for at least 5, 10, 15, 20, 30, 45, 60 consecutive minutes or longer.

It will be appreciated that the measuring may be carried out at least twice a day, wherein each measurement event is affected in at least 10%, 20%, 30%, 40%, 50%, 60%, 70%, 80%, 90% or even 100% of all breaths over a time period of at least five minutes. The interval between the two measurement events is at least one hour, at least two hours, at least three hours, at least four hours, at least five hours, at least six hours or longer or intermediate rates.

According to some exemplary embodiments, a value of at least one respiration parameter of nasal respirations are determined (e.g., recorded) at block 14.

In some embodiments, a respiration parameter characterizes one or both of inhalation and exhalation, for example, for a time period which is of longer duration than a single respiration.

In some embodiments, each respiration includes features including for example, the respiration trace itself, volume, duration, and peak airflow for each portion of the respiration, where portions include, for example, inhalation and exhalation.

In some embodiments, respiration parameters include average magnitude of respiration feature/s and/or, peak respiration feature/s and/or variability of respiration feature/s over time.

According to a particular embodiment, the value of the respiration feature is a peak value of the feature or a mean peak value of the feature over the course of a predetermined number of respirations or over the course of a predetermined time (e.g., five minutes).

According to a particular embodiment, the value of the respiration feature is a coefficient of variation of the feature over the course of a predetermined number of respirations or over the course of a predetermined time (e.g., five minutes).

Exemplary respiration parameters include timing related parameters and volume related parameters. These may include, for a measured time period:
variability of the respiration measurement;
average and variability, for inhalation and/or exhalation, for one or more of; timing, peak airflow speed and volume.

The analyzing includes analyzing at least one, at least two, at least three, at least four, at least five, at least six or all of the following respiration parameters:
Breathing rate (e.g., number of inspirations per minute);
Inter-breath interval (e.g., average time between inhale onsets);
Inhale volume e.g., sum of airflow between inhale onset and offset (e.g., calculated as integral of the signal);
Exhale volume e.g., sum of airflow between exhale onset and offset (e.g., calculated as integral of the signal;
Tidal volume (e.g., average volume of air displaced per breath, which can be calculated as average inhale volume+average exhale volume);
Minute ventilation (e.g., volume of air displaced each minute, which may be calculated from breathing rate×average tidal volume); and/or
Duty cycle (e.g., proportion of breath that is inhaled-Standard deviation of inhale duration/average inhale duration.

Additional respiration parameters that may be analyzed include for example, as follows. It is noted that Volume and Duration appear to be useful parameters with significant predictive value. Pauses may also be analyzed, for example, to detect prevalence and/or length of such pauses.

According to a particular embodiment, for ruling in Parkinsons's Disease (or for determining whether the subject is at risk of having Parkinson's Disease) at least one, at least two or even each of the following respiration parameters are analyzed:
1. Duration of nasal inspiration;
2. Variability of nasal inhalations; and
3. Length of time between successive nasal inhalations.

In one embodiment, the parameter is breathing rate variation coefficient (SD of difference between inhale onsets/average difference between inhale onsets).

In another embodiment, the parameter is duty cycle inhale (average inhale duration/average inter-breath interval).

In still another embodiment, the parameter is duty cycle of inhale pause duration (average duration of pause after inhale/average inter-breath interval.

According to a particular embodiment, for ruling in Alzheimer's Disease, determining a severity of Alzheimer's Disease, or for determining whether the subject is at risk of having Alzheimer's Disease, parameters associated with exhalation are measured. Optionally, parameters associated with inhalation are also measured. In one embodiment a ratio of a parameter associated with inhalation: parameter associated with exhalation is determined.

Parameters associated with exhalation include a length of a nasal exhalation, an exhalation velocity and a time between two nasal exhalations (i.e. exhale pause).

In another embodiment, the parameter is duty cycle exhale (average exhale duration/average inter-breath interval).

In still another embodiment, the parameter is duty cycle of exhale pause duration (average duration of pause after exhale/average inter-breath interval.

According to a particular embodiment, for determining a severity of Parkinson's Disease at least one, at least two of the following respiration parameters are analyzed:
1. percentage of breaths with inhale pause; and
2. percentage of breaths with exhale pause.

According to a particular embodiment, for determining a severity of Alzheimer's Disease at least one, at least two of the following respiration parameters are analyzed:
1. duty cycle exhale; and
2. exhale duration.

According to some exemplary embodiments, the status of the neurodegenerative disease (NDD) is determined at block 96. The status is determined based on the valued obtained at block 94. It is noted that the status may include an indication of the status, but need not have a reliability of over 90%. In many uses, a reliability of, for example, 70%, 80% or higher or intermediate reliabilities (e.g., sensitivity) may be useful. For example, if the subject is being screened, a relatively low false negative rate, even with a relatively high false positive rate may be good. In another example, if a subject is known to have the NDD, detecting changes in NDD severity may be sufficient, as the absolute diagnosis is known.

A status of NDD may refer, for example, to NDD severity or a sub-classification of NDD. The severity status of NDD (e.g., Parkinson's Disease) may correlate with known scales of severity—e.g., Hochn and Yahr scale and the Unified Parkinson's Disease Rating Scale.

The disease status may be obtained by comparing the at least one respiration parameter value with a reference value, wherein a difference between the two is indicative of a status of the disease. Optionally, the reference value is a personalized value for the subject. Alternatively, the reference value is a group reference value—e.g., for people of similar age and/or health status.

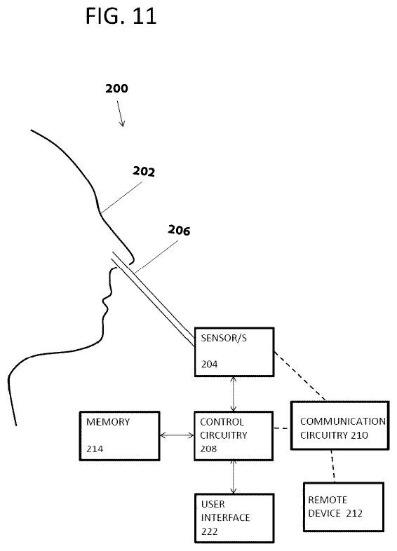

The comparison between the values can be made utilizing techniques such as reference limits, discrimination limits, or risk defining thresholds to define cutoff points and abnormal values. Comparison activities and/or data used (e.g., as reference) for such activities may be stored in a system, for example, as described herein with reference to FIG. 11.

In some embodiments, one or more threshold is selected and/or adjusted and/or determined depending on a desired status e.g., of a patient and/or patient group. For example, one or more threshold is selected and/or adjusted and/or determined based on desired specificity and/or sensitivity. In some embodiments, a one or more threshold is selected and/or adjusted and/or determined based on relative importance (e.g., in the assessment) of specificity and/or sensitivity.

In one embodiment, the reference value is the value of a normal control value which is derived from a subject (or group of subjects) not suffering from the NDD (e.g., Parkinson's Disease). According to a particular embodiment, an average value derived from a group of subjects known to not be suffering from the NDD is used as the control value. Such normal control values and cutoff points may vary based on whether a value is used from a single respiratory parameter or in a formula combined with values from other respiratory parameters into an index. Alternatively, the normal control level can be a database of respiratory parameter patterns from previously tested subjects.

When the value of the respiratory parameter of the tested subject is sufficiently similar (e.g., statistically significantly similar) to a reference value derived from a non-NDD subject (or group of subjects), it is indicative that the subject does not have the NDD. When the value of the respiratory parameter of the tested subject is sufficiently dissimilar (e.g., statistically significantly dissimilar) to a reference value derived from a non-NDD subject (or group of subjects), it is indicative that the subject has NDD.

In one embodiment, the reference value is the value of a control value which is derived from a subject (or group of subjects) suffering from the NDD. When the value of the respiratory parameter of the tested subject is sufficiently similar (e.g., statistically significantly similar) to a reference value derived from an NDD subject (or group of subjects), it is indicative of the NDD in the subject. When the value of the respiratory parameter of the tested subject is sufficiently dissimilar (e.g., statistically significantly dissimilar) to a reference value derived from an NDD subject (or group of subjects), it is indicative that the subject does not have the NDD.

In a first use, a subject who may be suspected of having NDD wears an NDD system (e.g., such as system 100, below), for a period of time, possibly in a clinical setting and optionally while performing a task. Such task and measurement may be managed by a computer. Optionally, measurements are taken with and without medication, so as to determine not only disease status, but also which/if certain medication can assist in improving disease status. Alternatively or optionally, such use may be for a subject where it is suspected that certain foods or drugs or activities cause disease-like symptoms. The trigger is applied and disease status is monitored. In a clinical setting, the system need not be mobile and may be, for example, include a nasal sensor connected by a cable (directly or indirectly) to an electronics box, such as a desktop or laptop computer.

In a second use, screening, no prior suspicion regarding the subject status is used. Instead, the system is applied to the subject for a period of time sufficient to provide an indication of disease status. This can be repeated for multiple subjects one after the other.

For screening and/or diagnosis, feedback re disease status may be provided to a user other than the subject.

### Exemplary System for Determining Disease Status

A disease status according to some embodiments refers to whether or not the subject is suffering from the disease (e.g., rule-in or rule-out).

In other embodiments, a disease status refers to a probability (a continuum or probability and/or severity and/or clinical significance) of the disease.

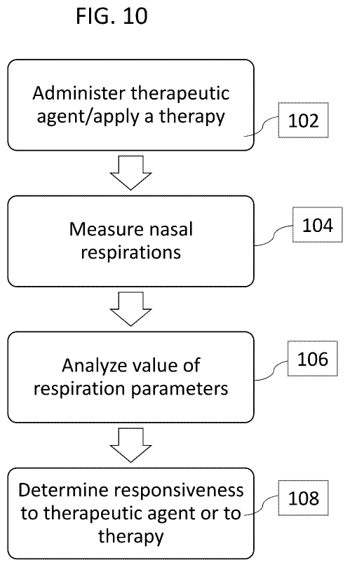

According to some exemplary embodiments, a disease status system, for example system 200 shown in FIG. 11 is used to determine disease status of a subject and/or for other methods, for example, as described with reference to FIGS. 9 and 10. The system may be programmed to control a UI and/or measurement, evaluation and/or communication circuitry as described in the methods. In some embodiments, nasal respiration is monitored by monitoring nasal airflow of at least one nostril of a subject using a device comprising one or more sensor 204 (e.g., a pressure or flow sensor).

In some embodiments, system 200 includes one or more additional sensor, e.g., for physiological measurement of subject. For example, one or more of a blood oxygenation sensor (e.g., located on a subject's finger), a temperature sensor, a cardiac cycle sensor. In some embodiments, an optical sensor detects and/or measures respiration (e.g., chest movement or nostril movement) and/or other subject parameters (e.g., other movements of the subject). Optionally, a smart watch or fitness sensor-type subsystem is used to collect physiological information. In some embodiments of the invention, sensor 204 includes an acceleration and/or other movement sensor to detect activity of the user (and optionally ignore measurements during movement). Alternatively, or optionally, sensor 204 includes an environmental sensor to detect environmental information such as audio (e.g., noise level), temperature, humidity and/or light. In some embodiments of the invention, sensors on a cellular telephone or other worn or hand-held electronic device is used to collect environmental and/or physiological information, for example, using sensing means and/or processing methods known in the art (e.g., microphone for sound, accelerometers for movement). It is noted that a typical cellular telephone has many sensors which may be repurposed, for example using methods know in the art, for collecting physiological and/or environmental data. Optionally, data collected by such sensors is used to calibrate the disease status, for example, for subjects where environmental factors (e.g., noise) increases disease-type behavior (e.g., tremors for Parkinson's Disease subjects). In some embodiments of the invention, a user can access their data from an app on their mobile phone and/or download it form a cloud location or other remote server.

According to some exemplary embodiments, a nasal respiration monitoring system receives the signals recorded at the measurements sites, and generates a disease indication, optionally in a form of index measurements. In some embodiments, the system presents the indication to a user.

According to some exemplary embodiments, the system comprises at least one circuitry, for example control circuitry 208, which processes the received signals. In some embodiments, the signal processing includes at least one of removing artifacts from the received signals and filtering of the received signals. In some embodiments, the received signals are processed using one or more algorithms formulas, and/or look-up tables (or other data) stored in a memory of the system, for example memory 214. Alternatively or additionally, the control circuitry processes the received signals using one or more algorithms, formulas and/or look-up tables and/or other data stored in a remote device 212. As can be seen, data processing may include multiple steps, including-signal processing to clear the signals, nasal parameter extraction from the optionally cleaned signals disease status determining from the extracted nasal parameter values and/or prediction of disease status and/or treatment suggestion, e.g., based on disease status. Additional processing in system 200 may include processing of non-nasal sensing, user interface management and process management. Each processing may be performed by separate processor in some embodiments. In others, two or more of these processing types are provided by a same circuitry (e.g., a processor). In an exemplary system the following processing loci may exist and one or more of them may be used: circuitry coupled to the sensors, a processor mounted on the subject's body, a mobile telephone, a cloud server. In some embodiments of the invention, the raw data or filtered data is used to directly indicate disease status, for example, by using a classifier trained on raw sensor data, rather than on extracted nasal flow parameters. Optionally, such a system is trained in two steps. First, a first classifier is generated using extracted nasal parameters. Once such first classifier is trained and/or validated, a new classifier can be trained on the raw data and receiving scoring of disease status using the trained classifier. Alternatively, a classifier may be trained on the raw data and using patient status indications. A potential advantage of the two-step method is that more data can be made available as the first classifier can provided many data points for a single subject, as, as noted herein, disease classification can be rapid.

According to some exemplary embodiments, a control circuitry of the system 200 analyzes the processed signals. In some embodiments, the analysis comprises at least one of calculating power and/or phase relationships between processed signals received from sensor 204, optionally using one or more signal features. Optionally, the control circuitry applies at least classifier or other model or parameter set (e.g., generated by a machine learning algorithm and/or a neural network classifier) on the one or more signal features to determine disease status, for example diagnosis and/or determine responsiveness of a subject to a medication/therapy. In some embodiments, the control circuitry analyses the processed signals using one or more algorithms stored in the memory of the system, for example memory 214. Alternatively or additionally, the control circuitry analyses the processed signals using one or more algorithms, formulas and/or look-up tables stored in a remote device 212. Optionally, the control circuitry analyses the processed signal taking into account the body and/or head posture of the subject.

According to some exemplary embodiments, the control circuitry optionally generates a confidence index. In some embodiments, the confidence index is calculated based on the analyzed signals and/or based on one or more indications stored in the memory of the system. In some embodiments, values of the confidence index indicate a degree of confidence of the calculated values of the disease status.

Additionally, in some embodiments, the disease status is generated using one or more subject-related indication stored in a memory of the system and/or in the remote device. In some embodiments, the subject-related indication comprise indication regarding at least one of, clinical state of the subject, subject age, subject BMI, medical history of the subject and/or drug regime of the subject.

In some embodiments of the invention, the subject-related indication comprises base line information about the patient, for example, a calibration cure or thresholds.

According to some exemplary embodiments, at least one or all of the disease status indications are communicated to a user of the system, for example to the subject himself and/or to a professional (and/or recorded in an electronic medical record or other logging system). In some embodiments, the indications are delivered using a user interface, for example user interface 222 or using a display operationally connected to the user interface 222. An example user interface includes one or more buttons and one or more indicator lights and/or a speaker. In another example. A UI includes a display, for example, a touch-sensitive display and/or speakers and/or a microphone of a cellular telephone. UI 222 generally also includes circuitry to present and receiving information and actions.

According to some exemplary embodiments, the system 202 comprises at least one communication circuitry 210 configured to transmit and/or to receive signals from at least one remote device, for example a remote device located at a distance larger than 1 meter from the system 202, a remote device located outside a room where the system 202 is located, a remote server, a cloud storage device, a remote database. In some embodiments, the at least one communication circuitry 210 is configured to transmit and/or receive wireless signals from the remote device 226, for example Bluetooth signals, Wi-Fi signals, and/or cellular signals.

According to some exemplary embodiments, the control circuitry is configured to process and/or to generate the information flow indication, using one or more algorithms stored in the remote device. In some embodiments, the control circuitry transmits the signals received from the sensors or indications thereof to the remote device, and received processed signals or the information flow indication from the remote device. Optionally the processing and/or the generation of the information flow indication is performed in the remote device 212 using one or more algorithms stored in the remote device 212.

In one embodiment, the nasal sensors may be on a strap that goes from the back of a subject's head to under the nostrils, where the sensors 204 may be located. Processing electronics may be at the nape of the neck and/or attached by a cable to such location.

### Exemplary Classifier

For example, in some embodiments, a logistic regression classifier is constructed based on all or subset of respiration parameters described in this document, including nasal respiration parameters such as breathing rate, an inter-breath interval, an inhale volume, an exhale volume, a tidal volume, a minute ventilation and a duty cycle. Where, in some embodiments, the classifier is constructed by choosing a cutoff value and classifying inputs with probability greater than the cutoff as one class, below the cutoff as the other. In some embodiments, the classifier detects a severity of the NDD (e.g., Parkinson's Disease or Alzheimer's Disease) and/or predicts responsiveness to drug therapy (e.g., dopamine or dopamine analogue for Parkinson's) or neurofeedback therapy.

For example, in some embodiments, a classifier is constructed using an alternative machine learning technique. For example, one or more of Perceptron, Naive Bayes, Decision Tree, K-Nearest Neighbor, Artificial Neural Networks/Deep Learning, and Support Vector Machine.

It should be noted that data can be stored in blocks, for example, 5 minute blocks of data and then prediction and/or training can use as many blocks as desired. As shown below, the classifier shows good results already with 30 minutes of breathing data. The loaded user data may include any one of the extracted nasal respiration parameters listed herein.

In brief a neural network classifier is optionally used, which may be trained, for example, with training data included 95% (n−1) of the disease and a balanced control group, and the testing consist 1 control and 1 disease. As preprocessing the raw data is optionally z-scored, and then divided into 5 minutes intervals. Outliers/intervals are optionally excluded. In some embodiments of the invention, the respiratory parameters per block are calculated and then averaged together.

In some embodiments, input/s to a classifier includes respiration parameter/s (e.g., at least one, two three, four, five, six or all of the following parameters: breathing rate, an inter-breath interval, an inhale volume, an exhale volume, a tidal volume, a minute ventilation and a duty cycle.

Optionally, in some embodiments, inputs to the classifier include the subject's state of health (or activity, such as walking) with respect to expected effect on the subject's physiological breathing apparatus. For example, subjects having respiration related conditions e.g., asthma, emphysema, pneumonia and/or conditions likely to affect respiration e.g., heart disease, in some embodiments, are assessed using different respiration parameter/s and/or using a portion of classifier which has been generated using respiration parameter data for this type of subject.

For example, in some embodiments, (e.g., where respiration volume is likely to be affected by a medical condition of the subject and/or an age of the subject) volume respiration parameters are normalized before use in assessment of the subject.

In some embodiments, output/s of a classifier include a probability that the subject is has a likelihood of the disease. In some embodiments, output/s of classifier include an indication regarding the subject responding to therapy, for example, based on responsiveness of other subjects with similar nasal parameters. Optionally or additionally, the classifier outputs an disease severity score, as a number on a scale.

In some embodiments, the classifier determines a probability that a subject has the disease (e.g., Parkinson's Disease or Alzheimer's Disease) using one or more respiration parameter including in some embodiments, only nasal respiratory parameters and, in some embodiments, both nasal respiratory and oral respiratory parameters. Where, in some embodiments, different parameters are weighted by the classifier.

Exemplary Process for Determining Responsiveness of a Subject Suffering from Parkinson's Disease to a Therapy

Reference is now made to FIG. 10, depicting a process for determining responsiveness of a Parkinson's Disease subject to a treatment, according to some exemplary embodiments of the invention. This process is particularly useful for identifying personalized treatments for a subject. The process may be useful for identifying combinations of agents for the treatment of Parkinson's Disease (e.g., dopamine+additional agent).

According to some exemplary embodiments, a first therapeutic agent or therapy is provided to a subject at block 102. The subject may already be taking a medication for the treatment of Parkinson's (e.g., dopamine or an analogue thereof)—i.e., as second therapeutic agent. The therapeutic agent may be an agent known to be generally useful in managing Parkinson's Disease (e.g., FDA approved drug). In one embodiment, the agent which is tested is not dopamine or dopamine analogue. It will be appreciated that the process described herein may be useful for determining therapeutic effect of candidate agents, whose activity is yet to be determined.

The present application also includes new dosage protocols for existing drugs, for example, timing personalized according to ongoing measurements. This may result in on-demand taking of a dosage and/or in planning new dose regimens for a subject, for example, based on a typical reaction to a drug and/or allowed maximum blood levels, a new regimen may be planned per subject which provides increased anti-Parkinson's Disease activity when needed.

Nasal respirations are measured at block 104. Measurements may occur simultaneously with the start of treatment or after a predetermined amount of time such that a therapeutic agent brings about the required effect. Alternatively, measurements may occur throughout a treatment protocol (e.g., during a breathing protocol). Alternatively or optionally, measurement starts before treatment so as to collect baseline state information.

Values of nasal respiratory parameters are analyzed at block 106. Exemplary parameters of nasal parameters are described herein above.

According to some exemplary embodiments, the responsiveness of the subject to the therapeutic agent/therapy is determined at block 108. The responsiveness is determined based on the valued obtained at block 106.

The responsiveness may be determined, for example, by comparing the at least one respiration parameter value with a reference value, wherein a difference between the two is indicative of responsiveness.

The comparison between the valued can be made utilizing techniques such as reference limits, discrimination limits, or risk defining thresholds to define cutoff points and abnormal values.

In one embodiment, the reference value is the value derived from the subject prior to administration of the therapeutic agent/therapy. The reference value may be obtained immediately prior to administration of the therapeutic agent or may be obtained on a different occasion.

In some embodiments of the invention, when the value of the respiratory parameter of the tested subject is sufficiently dissimilar (e.g., statistically significantly similar) to a reference value derived from the subject prior to administration of the medication/therapy, it is indicative that the treatment is efficacious. When the value of the respiratory parameter of the tested subject is similar (e.g., statistically significantly similar) to a reference value derived from the subject (prior to administration of the medication/therapy), it is indicative that the treatment is not efficacious.

In one embodiment, the reference value is the value of a control value which is derived from a different subject (or group of subjects) suffering from Parkinson's Disease. When the value of the respiratory parameter of the treated subject is sufficiently similar (e.g., statistically significantly similar) to a reference value derived from a Parkinson's Disease subject (or group of subjects), it is indicative that the treatment is not efficacious. When the value of the respiratory parameter of the treated subject is sufficiently dissimilar (e.g., statistically significantly dissimilar) to a reference value derived from a Parkinson's Disease subject (or group of subjects), it is indicative that the medication/therapy is efficacious. In one example, the group of reference subjects is selected based on one or more of gender, BMI, age and clinical/diagnostic information (such as questionnaire data). Optionally or additionally, the reference group is selected based on similarity of nasal parameters. Optionally, the groups are selected based on a post-hoc analysis showing which groups of subjects best (or good enough) predict. In one example, after generating a Parkinson's Disease classifier based on a set of, for example, 1000 subjects, a series of classifiers are generated using various subsets of the subjects and the subset which provides a best prediction is determined to be a good reference group. This subset may also be used to define similarity of nasal parameters. Other methods of optimization and subset selection may be used as well.

In one embodiment, the reference value is the value of a control value that is derived from a subject or group of subjects not suffering from Parkinson's Disease. When the value of the respiratory parameter of the treated subject is sufficiently similar (e.g., statistically significantly similar) to a reference value derived from a non-Parkinson's Disease subject (or group of subjects), it is indicative that the treatment is efficacious. When the value of the respiratory parameter of the treated subject is sufficiently dissimilar (e.g., statistically significantly dissimilar) to a reference value derived from a non-Parkinson's Disease subject (or group of subjects), it is indicative that the medication/therapy is non-efficacious.

It is noted that for the methods of FIGS. 9 and 10, as well as other methods described herein, other than nasal parameters may be used (e.g., sensor 204 is replaced by a different sensor or input type) and the methods described herein applied on non-nasal information. Alternatively or optionally, additional sensors which also provide Parkinson's Disease indication (e.g., diagnosis, staging) may be used, and the combined data from multiple sensors may be used to provide a potentially more accurate Parkinson's Disease status.

A particular potential advantage of nasal information for providing indications relating to neurodegenerative diseases associated with damage to the anterior olfactory nucleus (AON) is that breathing is under direct neural control and what is measured is changes in the breathing due to such control, rather than what is effectively noise in intended movements. Another potential advantage for nasal measurements is that while the measured signal is simpler (and similar across people and activities), the number of parameters is not small and can be focused all on the same activity, potentially leading to better results when building a classifier or applying other machine learning methods.

Furthermore, nasal respiration is a core function, as compared to, for example, movements. Another particular potential advantage of nasal measurements is that they may be less affected by movements of a subject (e.g., due to activity, such as computer or mobile device use). Another particular potential advantage of nasal measurements is that breathing may be more easily trained and/or controlled than fidgeting. Another potential advantage of nasal measurements is that they may be more difficult to fake and/or unintentionally control. Another potential advantage of nasal measurements is that measurement may be faster and/or include less variance, this may result from any of the advantages noted herein. Another potential advantage of nasal measurement is that they can be used for any sedentary activity (optionally being normalized to an indication of neural or other activation level, such as pulse or heart rate variability).

According to some exemplary embodiments, a control circuitry optionally generates a confidence index. In some embodiments, the confidence index is calculated based on the analyzed signals and/or based on one or more indications stored in the memory of the system. In some embodiments, values of the confidence index indicate a degree of confidence of the calculated values of the responsiveness of the subject to the therapy and/or on a change in disease status.

Additionally, in some embodiments, the index of responsiveness is generated using one or more subject-related indication stored in a memory of the system and/or in the remote device. In some embodiments, the subject-related indication comprise indication regarding at least one of, clinical state of the subject, subject age, subject BMI, medical history of the subject and/or drug regime of the subject. UI 222 may be used to allow a user to input information regarding the tested therapy—e.g., dose, timing, regimen, number of times user has been exposed to the therapy.

According to some exemplary embodiments, the responsiveness index of the subject to the therapy are communicated to a user of the system, for example to the subject himself and/or to a professional. In some embodiments, the indications are delivered using a user interface, for example user interface 222 or using a display operationally connected to the user interface 222.

### Device for Measuring Nasal Respirations

In some embodiments, the device is a sensor, for measurement of respiration e.g., of nasal airflow of the subject. In some embodiments, the sensor/s include a spirometer. In some embodiments, airflow sensor/s are fluidly connected to a cannula or probe which is placed within the subject's nasal passageway. In some embodiments of the invention, an airflow sensing system such as sold by sniff logic LTD of Tel-Aviv, Israel, may be used.

In some embodiments of the invention, measurements of nasal airflow are taken at 6 Hz. Higher or lower frequencies may be used.

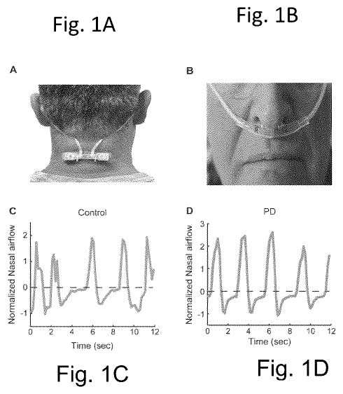

In some embodiments, the device comprises at least two independent sensors, one for measuring nasal airflow in the right nostril and one for measuring nasal airflow in the left nostril. The device may comprise a left nostril pressure probe which is configured to be inserted into a left nostril of a subject, and includes a left-nostril-pressure tube that is configured to transmit a left-nostril pressure wave from the left nostril to the left-nostril pressure sensor; a right-nostril pressure probe, which is configured to be inserted into a right nostril of the subject, and includes a right-nostril-pressure tube that is configured to transmit a right-nostril pressure wave from the right nostril to the right-nostril pressure sensor. An exemplary signal which can be recorded using such a device is shown in FIGS. 1A-B. Separate nostril sensors may be used, for example, for noise reduction, for example by averaging or selecting a best signal.

In some embodiments of the invention, the device further includes a pressure probe for analyzing oral pressure and/or oral respiratory patterns.

Alternatively or additionally, in some embodiments, temperature of nasal airflow is measured, for example by thermistor/s placed within the nasal air flow (and e.g., not in contact with the skin).

Alternatively or additionally, in some embodiments, a pneumotachometer is used to measure differential pressure of the nasal air flow. Where, in some embodiments, the differential pressure is converted into a voltage signal using a spirometer.

In an exemplary embodiment, nasal airflow is measured using a nasal cannula (e.g., 1103, Teleflex medical) linked directly to a spirometer (e.g., ML141, ADInstruments, H₂O resolution=15.6 μV). Where the spirometer, in some embodiments, converts airflow into a voltage signal. In an exemplary embodiment, the airflow voltage signal is amplified by an instrumentation amplifier (e.g., PowerLab 16SP Monitoring System, ADInstruments).

In some embodiments, data is collected by sampling the airflow voltage signal. In some embodiments, the airflow signal is sampled at 100-10,000 Hz, or 500-200 Hz, or at about 1000 Hz, or lower or higher or intermediate ranges or sampling rates. In an exemplary embodiment, sampling is at 1000 Hz.

In an exemplary embodiment, sampling is using LabChart software (ADInstruments).

An exemplary device that can be used to measure nasal respirations is disclosed in U.S. application Ser. No. 17/380,348 now US patent publication 2023/0028914A1, the contents of which are incorporated herein by reference.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Nasal Respiratory Parameters can be Used to Diagnose Parkinson's Disease

### Materials and Methods

Participants: 28 PD patients (age 66.35±6.6 years (manuscript stats are mean±s.d. unless stated otherwise); 2 women; 5 left-handed) and 33 age- and gender-matched healthy controls (age, 64.3±7.5 years; 4 women; 5 left-handed). PD diagnosis was confirmed both by clinical symptoms and by 18 F-DOPA PET/CT brain imaging.

Disease duration ranged from 1 to 29 years (median=6.5±5.6 years), and severity by MDS-UPDRS was Part I=11.5±5.6; Part II=14.35±5.6; Part III=30.5±11.6; Part IV=2.3±3.4, Total score=58.7±18.6; Hochn and Yahr H&Y stages (median±s.d.)=2±0.6. All PD patients were examined during the ON state. None of the patients reported OFF state during the 24-hour recordings. Two patients had genetic mutations associated with PD (one LRRK2 carrier, one GBA carrier). The groups did not differ in cognitive performance (MoCA PD=24.4±4.1, control=25.7±2.0, Mann-Whitney U=388.0, P=0.28). However, PD patients scored higher on the depression questionnaire (Beck PD=12.1±8.5, control=4.9±4.8, U=726.0, P<0.001) and had poorer olfaction (Sniffin′ Sticks total, PD=16.35±6.1, control=29.7±5.5, t (58)=8.85, P <0.001).

Measuring nasal airflow: A miniaturized version of a device previously described in detail was used (Kahana-Zweig, R. et al. PLOS One 11, e0162918 (2016). In brief, the Nasal Holter uses highly sensitive pressure sensors (SDP3x, Sensirion, Stäfa, Switzerland) and associated circuitry to convert pressure into flow at 6 Hz. The device measures 6.3×1.2 cm, weighs ˜22 g, is pasted to the nape of one's neck, and connected to the nose by nasal cannula (FIG. 1A). The device has onboard motion/position sensor allowing determination of sleep times. Data is stored onboard for later download. The device was applied during a clinic/lab visit and removed at the patient's home 24 hours later.

Data processing: Individual airflow (FIGS. 1C-D) was first z-scored, assigned to wake or sleep, and divided into 5-minute consecutive blocks. Null blocks (without respiration recording) and blocks that contained naps during the day were removed. 25 standard respiration features were extracted for each block. An unsupervised feature selection process was applied for multi-cluster data to rank-order the features. The top three that were best obtained with the Holter were selected. More specifically, the Holter provides a highly precise temporal trace, thus it is optimized to extract temporal features such as respiratory onsets, offsets, durations, and curve shapes. The three selected features were breathing rate variation coefficient (SD of difference between inhale onsets/average difference between inhale onsets), duty cycle inhale (average inhale duration/average inter-breath interval), and duty cycle of inhale pause duration (average duration of pause after inhale/average inter-breath interval).

Statistical analysis and evaluation: Distribution normality was tested using Kolmogorov-Smirnov tests. For normally distributed data, differences between groups using independent sample t-tests were tested, whereas for abnormally distributed data, non-parametric Wilcoxon rank-sum tests, with Bonferroni correction for multiple comparisons was applied. To classify PD, the Subspace Discriminant classifier, was applied using a leave-one-out cross validation method such that predictions were made on participants who were not in the learning set. For PD severity prediction, a linear regression model was used. To assess severity prediction, a Pearson's correlation was between PD outcome measures (MDS-UPDRS total score) and the result of the linear regression analysis was measured. Analyses were performed using JASP (version 0.17.2.1, JASP team) and MATLAB (version R2023a, MathWorks, Inc.).

### Results

### Daytime Nasal Airflow is Significantly Altered in PD

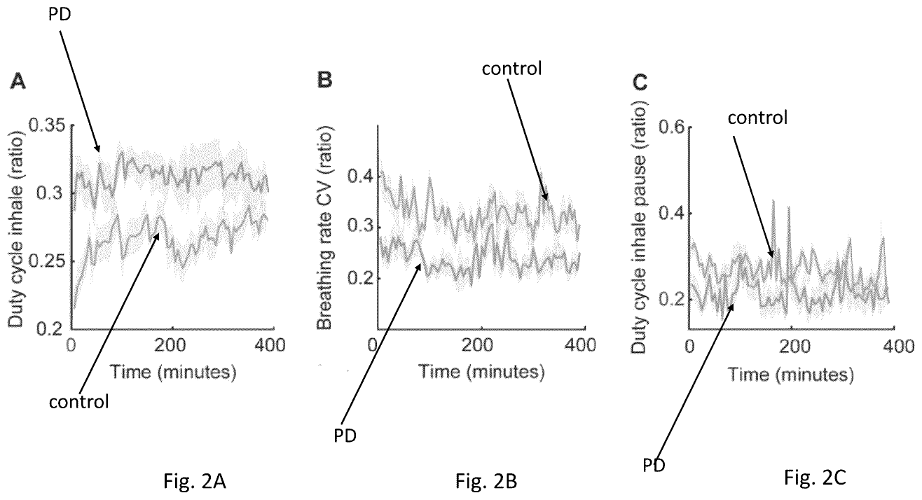

Initially, respiratory features from 28 PD patients and 33 controls, over 24 hours of recording were plotted. Significant differences between PD and controls were observed (FIGS. 2A-C). Although differences were significant across the recording period, they were greatest during the initial 30 minutes of recording, so this time window for subsequent experiments.

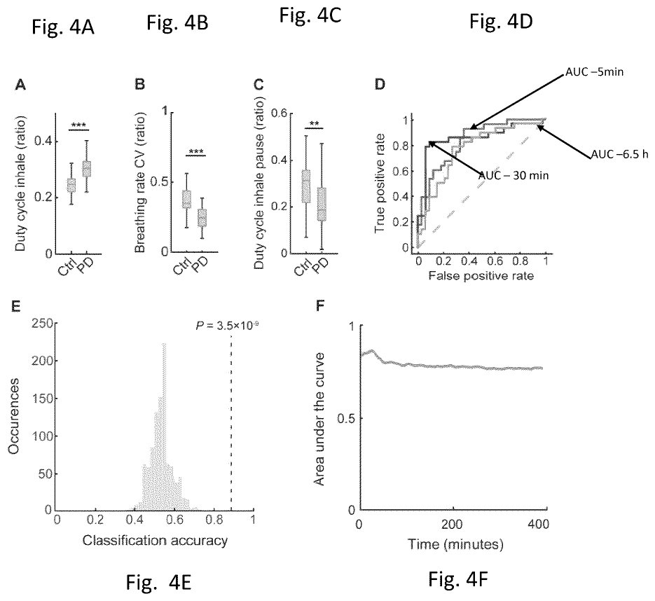

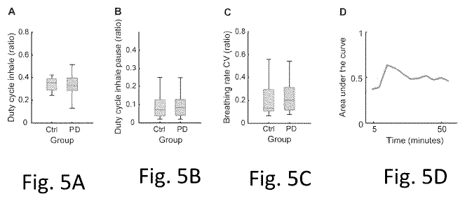

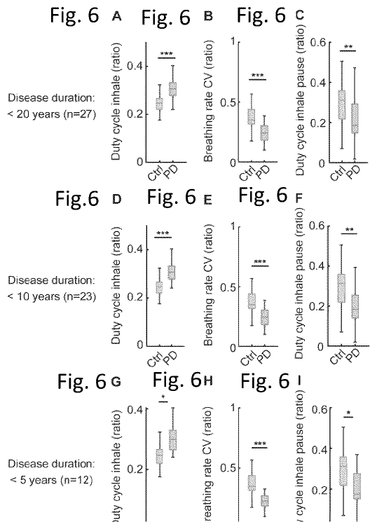

Differences were most pronounced in Duty Cycle Inhale (mean PD=0.3+0.07 (ratio), Control=0.23±0.06 (ratio), Wilcoxon rank-sum test, z=4.2, effect size Cliff's δ=0.63, P=6.9×10−5 (all manuscript P values Bonferroni corrected) and breathing rate variation coefficient (mean PD=0.26±0.11 (ratio), Control=0.38±0.13 (ratio), z=−4.3, effect size Cliff's δ=−0.64, P=5.3×10⁻⁵). In other words, PD breathing is characterized by longer and less variable nasal inhalations. Significant differences in duty cycle inhale pause were also observed (mean PD=0.2±0.1 (ratio), Control=0.29±0.1 (ratio), z=−3.06, effect size Cliff's δ=−0.45, P=0.006) (FIGS. 4A-C).

Whereas all the effects were pronounced during wake, similar differences were not observed in sleep (FIGS. 5A-D). Although most of the patients were Stage II, several patients were included in the study with extended disease duration. To assess their contribution to these results, the above depictions were recreated, restricting the cohort to disease duration equal/under 20 years (n=27), equal/under 10 years (n=23), and equal/under 5 years (n=12). As can be seen in FIGS. 6A-I, the differences between the groups remain highly significant even after removal of all patients with advanced and prolonged disease, such that even in the 5-year group significant effects were observed (Duty cycle inhale: mean PD=0.29±0.08, mean control=0.23±0.06, z=2.8, effect size Cliff's δ=0.6, P=0.02. Breathing rate variation coefficient: mean PD=0.26±0.1, mean control=0.38±0.1, z=−3.5, effect size Cliff's δ=−0.7, P=0.001. Duty cycle inhale pause: mean PD=0.2±0.1, mean control=0.29±0.1, z=−2.6, effect size Cliff's δ=−0.5, P=0.03) (FIGS. 6A-I).

### PD can be Classified Using Nasal Airflow Alone

In order to determine whether it is possible to classify PD using this data, the present inventors applied the Subspace Discriminant Classifier using a leave-one-out cross validation procedure, such that predictions were made on participants who were not in the learning set. Using only the first 5 minutes from each participant, the model achieved an area under the curve (AUC) of 0.83 (FIG. 4D), providing for 74% accuracy, 68% sensitivity (19 of 28) and 79% specificity (26 of 33). The present inventors further explored whether the accuracy improves by using a longer period of signal from each participant. To do so, they computed the PD prediction score for each 5-minute block and used the mean PD prediction score as the final diagnostic score.

The PD prediction score ranges between 0 to 1, where a score above 0.5 classifies the subject as PD. Performance improved as data was added up to 30 minutes of recording, where an AUC of 0.85 (FIG. 4D), 87% accuracy, 79% sensitivity (22 of 28) and 94% specificity (31 of 33) was obtained. To assess the significance of this result, a bootstrap analysis was conducted in which the labels of participants (PD/Control) were randomly shuffled 1000 times, and the classification was reconducted. It was observed that the chance probability to obtain this outcome is 1 in 3.5×10⁹ (bootstrapped z=5.7, FIG. 4E).

Using additional data beyond 30 minutes did not improve performance, and using all the available data resulted in 0.76 AUC (FIG. 4D 66% accuracy, 39% sensitivity (11 of 28) and 88% specificity (29 of 33)), which was not significantly different from the first 30 minutes alone (AUC differences=0.09, χ₂=2.7, P=0.1).

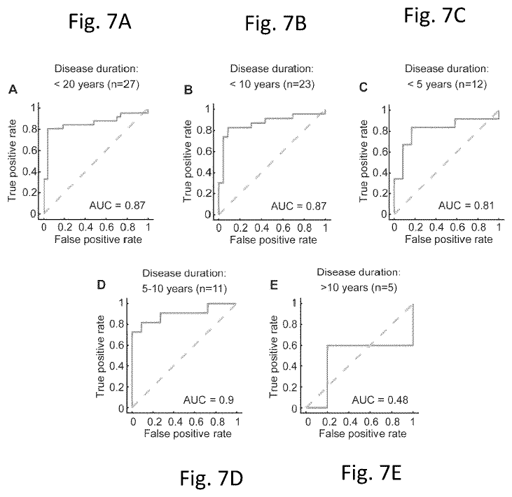

To ask whether the first 30 minutes were privileged, the data was parsed into 30-minute windows, with a sliding window of 5 minutes, and accuracy was compared across these selections. It was observed that the first 30 minutes were slightly but significantly better than any other 30 minutes in the data (first 30 minutes=87%, all other 30 minute windows=63±0.05%, z=4.53, P=2.9×10⁻⁶). As before, to assess the reliance of this result on disease duration, the above analysis was recreated, restricting the cohort to disease duration≤20 years (n=27), ≤10 years (n=23), and ≤5 years (n=12). As can be seen in FIG. 7A, the classifier remains highly effective and significant even within the group of five years or less from diagnosis (n(PD)=12, accuracy 18 of 24 (75%), sensitivity: 10 of 12 (83%), specificity: 8 of 12 (66%), AUC 0.81) (FIGS. 7A-E). Finally, in contrast to this remarkable classification during wake, it was not possible to classify PD during sleep using these method and recordings (FIGS. 5A-D).

PD Severity can be Predicted from Nasal Airflow Alone

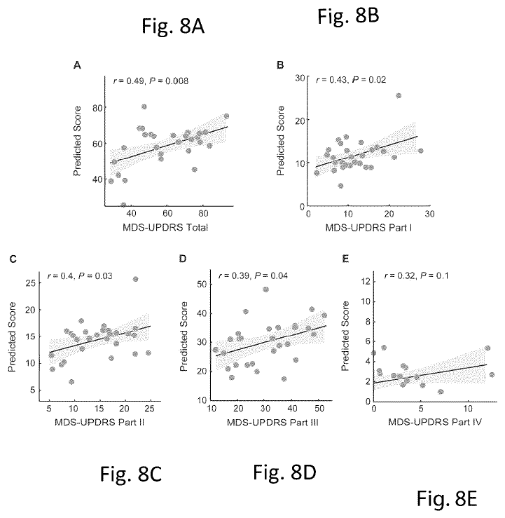

To explore whether the present model could predict disease severity (even though it detects PD regardless of disease duration or severity within the present cohort), the model scores were regressed against the Movement Disorder Society-Unified Parkinson's Disease Rating Scale (MDS-UPDRS) score for each participant. Based on the findings that the first 30 minutes are the most valuable for classification purposes, the present inventors performed regression analysis for the features within these first 30 minutes. They performed a linear regression analysis with leave-one-out cross validation, such that predictions were made on participants who were not in the learning set. As expected, they did not observe a significant correlation between the predicted and the observed total score, or any of the sub-scores (all r<½0.34½, all P>0.1). To determine whether two nasal airflow features (percentage of breaths with inhale pause and percentage of breaths with exhale pause) can predict disease severity, the same analysis scheme was applied as above. A moderate but highly significant correlation between the predicted and the observed total score (Total score: r=0.49; P=0.008) was observed (FIG. 8A). Similar correlations emerged between the predicted and the observed three subparts (Part I: r=0.43, P=0.02; Part II: r=0.4, P=0.03, Part III: r=0.39, P=0.04) (FIGS. 8C-E), implying that the model can predict disease severity. Only correlation with Part IV, that reflects rare therapy complications, was non-significant (Part IV: r=0.32, P=0.1) (FIG. 8E), as a majority of patients scored Zero on this part.

### Example 2

### Nasal Respiratory Parameters can be Used to Diagnose Parkinson's Disease and Alzheimer's Disease

### Materials and Methods

33 individuals with PD (mean age: 65.8±7.2, 4 women), 33 matched healthy controls (mean age: 64.2±7.5, 4 women) and 21 patients with Alzheimer's disease (AD) (mean age: 73.6 ±6.5, 12 women). All individuals with Alzheimer's Disease (AD) were at the early symptomatic stage, clinically classified as mild AD. Importantly, their diagnosis was biologically confirmed based on established AD biomarkers (CSF-based evidence of amyloid and tau pathology). Measuring nasal airflow and data processing was carried out as described in Example 1.

### Results

As illustrated in FIGS. 12A-C, Parkinson's patients could be distinguished from healthy patients on the basis of nasal inhale respiration parameters.

Breathing rate of Parkinson's patients was reduced compared to healthy subjects (FIG. 12A). Duty cycle inhale of Parkinson's patients was increased compared to healthy subjects (FIG. 12B). Duty cycle inhale pause was decreased in Parkinson's patients as compared to healthy subjects.

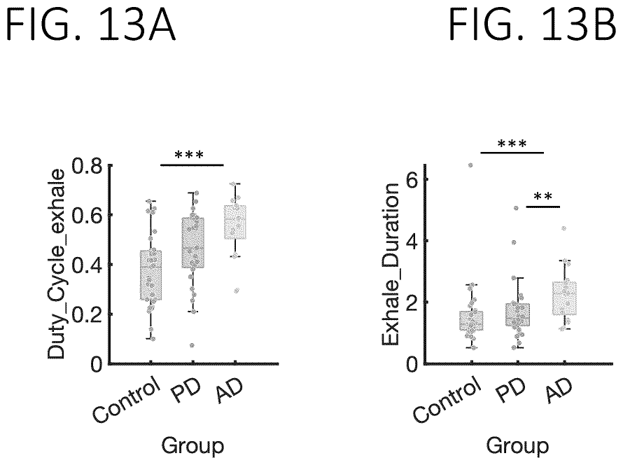

As illustrated in FIGS. 13A-B, both Parkinson's patients and Alzheimer's disease patients could be distinguished from healthy patients on the basis of nasal inhale respiration parameters.

Duty cycle exhale was increased in Parkinson's patients as compared to healthy subjects. Duty cycle exhale was increased in Alzheimer's patients as compared to healthy subjects. Furthermore, duty cycle exhale was increased in Alzheimer's patients as compared to Parkinson's patients (FIG. 13A).

Exhale duration was increased in Parkinson's patients as compared to healthy subjects. Exhale duration was increased in Alzheimer's patients as compared to healthy subjects. Furthermore, exhale duration was increased in Alzheimer's patients as compared to Parkinson's patients (FIG. 13B).

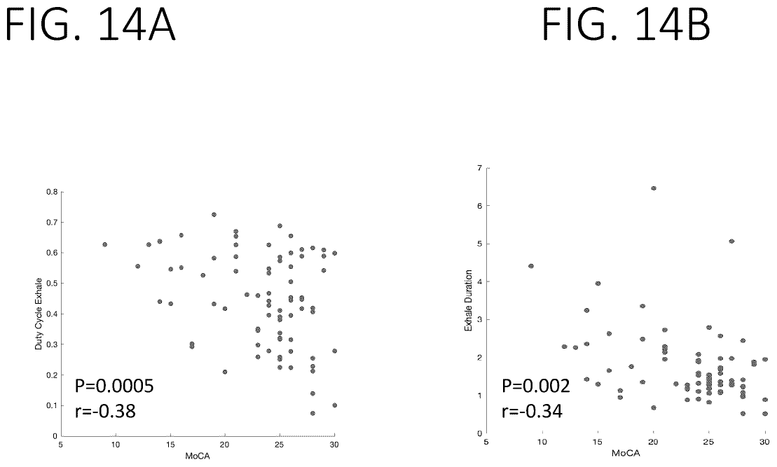

As illustrated in FIGS. 14A-B, exhale data correlated with MoCA score (severity).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for generating an indication for Parkinson's Disease in a subject comprising:
measuring, whilst the subject is awake, air flow in the nose of the subject during a plurality of nasal respirations using a sensor;
receiving, by a device, data associated with said air flow measurements received from said sensor;
evaluating at least one respiration parameter received from said data, wherein said respiration parameter comprises at least one of a duration of a breath, a variability of respiration rate and a pausing characteristic of said breath; and
generating an indication for Parkinson's Disease of the subject based on said at least one respiration parameter.

2. The method of claim 1, wherein said measuring comprises measuring airflow in said at least one nostril during at least 10% of the breaths of the subject over a period of at least ten minutes.

3. The method of claim 1, wherein said evaluating is effected using data which is retrieved whilst the subject is carrying out a predefined activity.

4. The method of claim 1, further comprising measuring, a time between two nasal inhalations, an inhalation velocity, an exhalation velocity and a time between two nasal exhalations.

5. The method of claim 1, wherein said generating an indication comprises determining whether the subject has Parkinson's Disease, is at risk of having Parkinson's Disease or determining a severity of Parkinson's Disease.

6. A system for generating an indication for Parkinson's Disease in a subject comprising:
receiving circuitry configured to receive a measurement signal including measurements of a plurality of respirations from a sensor configured to sense a respiration of the subject whilst awake;
determining circuitry configured to determine values of one or more respiration parameter from said measurement signal, said respiration parameter comprises at least one of a duration of a breath, a variability of respiration rate and a pausing characteristic of said breath;
evaluating circuitry configured to diagnose said subject, based on said values of one or more respiration parameter to provide an indication of the Parkinson's Disease.

7. The system of claim 6, wherein said one or more respiration parameter comprises a temporal nasal respiration parameter.

8. The system of claim 6, wherein said evaluation circuitry includes a classifier trained to translate breathing data into a Parkinson's Disease diagnosis.

9. The system of claim 6, wherein said Parkinson's Disease diagnosis comprises a disease sub-classification or a disease severity score.

10. The system of claim 6, wherein said evaluation circuitry is configured to compare said value of said at least one respiration parameter with a reference value, wherein a difference between said value of at least one respiration parameter of said subject and said reference value is indicative of the disease diagnosis.

11. The system of claim 6, comprising memory storing therein at least one reference value personalized for the subject.

12. The system of claim 6, comprising at least one additional sensor configured to sense environmental data and/or physiological data of the subject and wherein said evaluation circuitry is configured to take such data into account in said evaluating.

13. A system for generating an indication of severity of a neurodegenerative disease associated with damage to the anterior olfactory nucleus (AON) in a subject comprising:
receiving circuitry configured to receive a measurement signal including measurements of a plurality of respirations from a sensor configured to sense a respiration of the subject whilst awake;
determining circuitry configured to determine values of one or more respiration parameter from said measurement signal;
evaluating circuitry configured to diagnose said subject, based on said values of one or more respiration parameter to provide a an indication of severity of the neurodegenerative disease associated with damage to the AON.

14. The system of claim 13, wherein said one or more respiration parameter comprises a temporal nasal respiration parameter.

15. The system of claim 13, wherein said neurodegenerative disease is PD.

16. The system of claim 15, wherein said temporal respiration parameter comprises at least one of a duration of a nasal inhalation, a duration of a nasal exhalation, a time between two nasal inhalations, an inhalation velocity, an exhalation velocity and a time between two nasal exhalations.

17. The system of claim 13, wherein said neurodegenerative disease is AD.

18. The system of claim 17, wherein said temporal respiration parameter comprises at least one of a duration of a nasal exhalation, an exhalation velocity and a time between two nasal exhalations.

19. The system of claim 13, wherein said evaluation circuitry includes a classifier trained to translate breathing data into a neurodegenerative disease subclassification or a disease severity score.

20. The system of claim 13, wherein said evaluation circuitry is configured to compare said value of said at least one respiration parameter with a reference value, wherein a difference between said value of at least one respiration parameter of said subject and said reference value is indicative of the disease diagnosis.
